(19) United States
(12) Patent Application Publication
(10) Pub. No.: US 2023/0120871 A1
(43) Pub. Date: Apr. 20, 2023

(54) SYSTEM FOR DELIVERING TARGETED CONTENT TO UNIQUE USER PERSONA GROUPS

(71) Applicant: Texas Medical Center, Houston, TX (US)

(72) Inventors: Desh Mohan, Houston, TX (US); Tatiana Fofanova, Houston, TX (US); Katelin Cherry, Houston, TX (US)

(21) Appl. No.: 17/907,396

(22) PCT Filed: Mar. 25, 2021
(86) PCT No.: PCT/US2021/024087

§ 371 Date: Sep. 26, 2022

Related U.S. Application Data
(60) Provisional application No. 63/000,636, filed on Mar. 27, 2020.

(51) Int. Cl. G16H 10/60

(52) U.S. Cl. CPC G16H 10/60

(57) ABSTRACT

Described are platforms, systems, and methods for providing targeted content based on a determined persona. In one aspect, a method comprises receiving, from a user-interface, user-generated data comprising readiness, values, or personality, of a user; processing the user-generated data through a persona clustering model to determine a persona from a plurality of personas for the user, the persona clustering model trained with previously received user-generated data for a plurality of other users; determining targeted content for the user based on the persona; and providing, to the user-interface, the targeted content customized for the user.

### CROSS-REFERENCE

This application claims the benefit of U.S. Provisional Application No. 63/000,636, filed Mar. 27, 2020, which is hereby incorporated by reference in its entirety herein.

### BACKGROUND

Machine learning is a subset of artificial intelligence where computer algorithms are programmed to learn on their own from new and large sets of data (“Big Data”) in order to perform certain actions. These self-driving systems adapt and change their actions based on historical data and patterns, and the more crucial information they gather, the more accurate they become.

### SUMMARY

Embodiments of the present disclosure are generally directed to a system that provides targeted content customized to a user based on a persona determined by processing user-generated data through a persona clustering model trained with previously received user-generated data for a plurality of other users

With so few patients engaging in high quality conversations about their end of life healthcare preferences, they often receive unwanted care which is a huge emotional stressor on families that costs payors billions of dollars annually. Advance care planning conversations, which are the dedicated exploration of a patient's values and healthcare objectives, have demonstrated efficacy in addressing this issue. However, physicians lack the time and training to have them.

Physician discomfort with the end-of-life conversation has resulted in an epidemic of unwanted care, costing billions of dollars annually. In the current standard of care, patients' healthcare decisions are not being fulfilled at the end of life even when properly documented. The current standard of care involves the completion of highly simplistic forms called “advanced directive,” which are hardly referenced by physicians and are ineffective. In current practice, advance directives are usually completed by pen and paper. They are difficult to interpret for both users and physicians, and do not cover all the care options that need to be considered. When filled out, these advance directives are often incomplete or incorrectly completed since there is not a robust system of verification. Thus, they are often of low-value to medical decision makers and physicians.

As such, there is an opportunity for digital health systems to play a more prominent role in educating users on advance care planning. Current digital health systems do not have the capability of receiving user-information to recommend targeted material based on their responses. Given that the advance care planning conversation is personal and unique to every user, there needs to be a personalized approach to content delivery.

The described targeted content system provides targeted content that is simplified and easy to understand. In some embodiments, the system immediately prompts users for input regarding their care preferences related to the provided content. This input can be employed to, for example, generate a high-value advance directive populated with the received input. In some embodiments, the described system verifies the completeness of the generated advance directive to ensure that the document is usable and of high-value to the user.

Accordingly, in one aspect, described herein are computer-implemented methods for providing targeted content based on a determined persona. The methods are executed by one or more processors. The methods comprise: receiving, from a user-interface, user-generated data comprising readiness, values, or personality, of a user; processing the user-generated data through a persona clustering model to determine a persona from a plurality of personas for the user, the persona clustering model trained with previously received user-generated data for a plurality of other users; determining targeted content for the user based on the persona; and providing, to the user-interface, the targeted content customized for the user. In some embodiments, the persona clustering model is retrained with the determined persona and the received user-generated data. In some embodiments, the persona is determined based on the readiness, the values, or the personality, provided for the user. In some embodiments, the targeted content is determined based on key motivators to engage in advance care planning according to the determined persona. In some embodiments, the targeted content comprises education regarding advance care planning for the user, wherein the user can select care preferences. In some embodiments, the methods comprise: after providing the targeted content, receiving, from the user-interface, a selection of the care preferences; and persisting the selected care preferences to a data store. In some embodiments, the methods comprise: verifying the selection of care preferences, and generating an advanced directive based on the selection of care preferences. In some embodiments, verifying the selection of care preferences comprises verifying the completeness of the selection of care preferences and verifying a logical consistency of the selection of care preferences. In some embodiments, the methods comprise: providing, to the user-interface, the advanced directive determined based on the selection of care preferences; receiving, from the user-interface, a digital mark of the user for the advanced directive; and verifying the digital mark. In some embodiments, the methods comprise: receiving, via an application program interface (API) provided by an electronic medical records (EMR) provider, EMR data for the user; and processing the EMR data through the persona clustering model to determine the persona. In some embodiments, the EMR data comprises diagnoses, demographics, or visit history for the user. In some embodiments, the methods comprise: receiving, from the user-interface, the user-generated data for the other users; receiving, via the API provided the EMR provider, EMR data for the other users; and training the persona clustering model with the received user-generated data for the other users and the EMR data for the other users.

In another aspect, described herein are non-transitory computer-readable storage media coupled to one or more processors and having instructions stored thereon which, when executed by the one or more processors, cause the one or more processors to perform operations. These operations comprise: receiving, from a user-interface, user-generated data comprising readiness, values, or personality, of a user; processing the user-generated data through a persona clustering model to determine a persona from a plurality of personas for the user, the persona clustering model trained with previously received user-generated data for a plurality of other users; determining targeted content for the user based on the persona; and providing, to the user-interface, the targeted content customized for the user. In some embodiments, the persona clustering model is retrained with the determined persona and the received user-generated data. In some embodiments, the persona is determined based on the readiness, the values, or the personality, provided for the user. In some embodiments, the targeted content is determined based on key motivators to engage in advance care planning according to the determined persona. In some embodiments, the targeted content comprises education regarding advance care planning for the user, wherein the user can select care preferences. In some embodiments, the operations comprise: after providing the targeted content, receiving, from the user-interface, a selection of the care preferences; and persisting the selected care preferences to a data store. In some embodiments, the operations comprise: verifying the selection of care preferences, and generating an advanced directive based on the selection of care preferences. In some embodiments, verifying the selection of care preferences comprises verifying the completeness of the selection of care preferences and verifying a logical consistency of the selection of care preferences. In some embodiments, the operations comprise: providing, to the user-interface, the advanced directive determined based on the selection of care preferences; receiving, from the user-interface, a digital mark of the user for the advanced directive; and verifying the digital mark. In some embodiments, the operations comprise: receiving, via an API provided by an EMR provider, EMR data for the user; and processing the EMR data through the persona clustering model to determine the persona. In some embodiments, the EMR data comprises diagnoses, demographics, or visit history for the user. In some embodiments, the operations comprise: receiving, from the user-interface, the user-generated data for the other users; receiving, via the API provided the EMR provider, EMR data for the other users; and training the persona clustering model with the received user-generated data for the other users and the EMR data for the other users.

In another aspect, described herein are systems for providing targeted content customized for a user. These systems comprise: a user device; one or more processors; and a computer-readable storage device coupled to the one or more processors and having instructions stored thereon which, when executed by the one or more processors, cause the one or more processors to perform operations. These operations comprise: receiving, from the user-interface, user-generated data comprising readiness, values, or personality, of a user; processing the user-generated data through a persona clustering model to determine a persona from a plurality of personas for the user, the persona clustering model trained with previously received user-generated data for a plurality of other users; determining targeted content for the user based on the persona; and providing, to the user-interface, the targeted content customized for the user. In some embodiments, the persona clustering model is retrained with the determined persona and the received user-generated data. In some embodiments, the persona is determined based on the readiness, the values, or the personality, provided for the user. In some embodiments, the targeted content is determined based on key motivators to engage in advance care planning according to the determined persona. In some embodiments, the targeted content comprises education regarding advance care planning for the user, wherein the user can select care preferences. In some embodiments, the operations comprise: after providing the targeted content, receiving, from the user-interface, a selection of the care preferences; and persisting the selected care preferences to a data store. In some embodiments, the operations comprise: verifying the selection of care preferences, and generating an advanced directive based on the selection of care preferences. In some embodiments, verifying the selection of care preferences comprises verifying the completeness of the selection of care preferences and verifying a logical consistency of the selection of care preferences. In some embodiments, the operations comprise: providing, to the user-interface, the advanced directive determined based on the selection of care preferences; receiving, from the user-interface, a digital mark of the user for the advanced directive; and verifying the digital mark. In some embodiments, the operations comprise: receiving, via an application program interface (API) provided by an electronic medical records (EMR) provider, EMR data for the user; and processing the EMR data through the persona clustering model to determine the persona. In some embodiments, the EMR data comprises diagnoses, demographics, or visit history for the user. In some embodiments, the operations comprise: receiving, from the user-interface, the user-generated data for the other users; receiving, via the API provided the EMR provider, EMR data for the other users; and training the persona clustering model with the received user-generated data for the other users and the EMR data for the other users.

Particular embodiments of the subject matter described in this disclosure can be implemented so as to realize one or more of the following advantages. The described targeted content system employs psychometrics to identify a user's core values and motivators to determine a persona for the user. Once the user's persona is determined, content is tailored for the user to create an engaging experience. Users can explore their care options and select what is important to them to generate a comprehensive care plan that can easily be shared with loved ones and medical providers. For high-risk patients that may require a higher-touch follow-up, this persona data can be leveraged to optimize the conversation and significantly minimize labor costs relative to existing advanced care planning providers.

It is appreciated that methods in accordance with the present disclosure can include any combination of the aspects and features described herein. That is, methods in accordance with the present disclosure are not limited to the combinations of aspects and features specifically described herein, but also may include any combination of the aspects and features provided.

The details of one or more embodiments of the present disclosure are set forth in the accompanying drawings and the description below. Other features and advantages of the present disclosure will be apparent from the description and drawings, and from the claims.

### BRIEF DESCRIPTION OF THE DRAWINGS

A better understanding of the features and advantages of the present subject matter will be obtained by reference to the following detailed description that sets forth illustrative embodiments and the accompanying drawings of which:

FIGS. 1A-1C depicts various non-limiting example architectures of the described targeted content system;

FIG. 2 depict a flowchart of a non-limiting example process that can be implemented by embodiments of the present disclosure;

FIG. 3 depict another flowchart of a non-limiting example process that can be implemented by embodiments of the present disclosure;

FIG. 4 depicts a non-limiting example a computer system that can be programmed or otherwise configured to implement methods or systems of the present disclosure;

FIG. 5A depicts a non-limiting example environment that can be employed to execute embodiments of the present disclosure;

FIG. 5B depicts a non-limiting example application provision system that can be provided through an environment and employed to execute embodiments of the present disclosure; and

FIG. 5C depicts a non-limiting example cloud-based architecture of an application provision system that can be provided through an environment and employed to execute embodiments of the present disclosure.

### DETAILED DESCRIPTION

Described herein, in certain embodiments, are computer-implemented methods for providing targeted content based on a determined persona. The methods are executed by one or more processors. The methods comprise: receiving, from a user-interface, user-generated data comprising readiness, values, or personality, of a user; processing the user-generated data through a persona clustering model to determine a persona from a plurality of personas for the user, the persona clustering model trained with previously received user-generated data for a plurality of other users; determining targeted content for the user based on the persona; and providing, to the user-interface, the targeted content customized for the user.

Also described herein, in certain embodiments, are non-transitory computer-readable storage media coupled to one or more processors and having instructions stored thereon which, when executed by the one or more processors, cause the one or more processors to perform operations. These operations comprise: receiving, from a user-interface, user-generated data comprising readiness, values, or personality, of a user; processing the user-generated data through a persona clustering model to determine a persona from a plurality of personas for the user, the persona clustering model trained with previously received user-generated data for a plurality of other users; determining targeted content for the user based on the persona; and providing, to the user-interface, the targeted content customized for the user.

Also described herein, in certain embodiments, are systems for providing targeted content customized for a user. These systems comprise: a user device; one or more processors; and a computer-readable storage device coupled to the one or more processors and having instructions stored thereon which, when executed by the one or more processors, cause the one or more processors to perform operations. These operations comprise: receiving, from the user-interface, user-generated data comprising readiness, values, or personality, of a user; processing the user-generated data through a persona clustering model to determine a persona from a plurality of personas for the user, the persona clustering model trained with previously received user-generated data for a plurality of other users; determining targeted content for the user based on the persona; and providing, to the user-interface, the targeted content customized for the user.

### Certain Definitions

Unless otherwise defined, all technical terms used herein have the same meaning as commonly understood by one of ordinary skill in the art to which this disclosure belongs.

Unless otherwise defined, all technical terms used herein have the same meaning as commonly understood by one of ordinary skill in the art to which this invention belongs. As used in this specification and the appended claims, the singular forms “a,” “an,” and “the” include plural references unless the context clearly dictates otherwise. Any reference to “or” herein is intended to encompass “and/or” unless otherwise stated.

As used herein, the term “real-time” refers to transmitting or processing data without intentional delay given the processing limitations of a system, the time required to accurately obtain data and images, and the rate of change of the data and images. In some examples, “real-time” is used to describe the presentation of information obtained from components of embodiments of the present disclosure.

As used herein, the term “advance care planning” includes the in-depth exploration of the care and values that are important to a person if they become seriously ill or towards the end of life, and they are not able to make decisions for themselves. When people successfully go through an advance care planning conversation and share their wishes with their loved ones and physicians, they are more likely to receive the care that they want if they end up in a critical condition. This conversation may also lead to filling out an advance directive, which is legal documentation detailing their wishes. This conversation is sometimes facilitated by physicians for their patients, but physicians often don't initiate these conversations early enough due to lack of time and training. Thus, there is a need for a digital solution to help facilitate this conversation. When it comes to the sensitive subject matter covered in the advance care planning conversation, each person has different motivators to engage in the conversation. Some of these motivators include reducing family burden, maintaining personal independence, and ensuring financial security. When facilitators typically guide people through these conversations, they are able to gauge the motivators of an individual and tailor the educational material accordingly. It is important for digital solutions to facilitate advance care planning to be able to similarly gauge individual motivators and tailor content accordingly.

As used herein, the term “persona” includes the classification group for a segment of individuals that are motivated by similar factors. As detailed above, some of the motivators for a persona group may include reducing family burden, maintaining personal independence, or ensuring financial security. The described targeted content system processes user-generated input (e.g., user values and personality) and optionally, EMR-input (e.g., demographics and diagnoses), through a trained model to group an individual into a particular persona group.

As used herein, the term “targeted content” includes content personalized for each defined persona within the described targeted content system as users are more likely to engage in advance care planning due to particular motivators. The described system employs key motivators (e.g., family, independence, financial security, and so forth) to engage in advance care planning. For example, based on a user's identified persona, targeted content that includes personalized education is provided to each user of the system. This targeted content is critical to being able to deliver a personalized advance care planning experience that patients require.

### Targeted Content System

In some embodiments, the described targeted content system delivers targeted advance care planning content to users based on user-provided data or data from the user's medical record. In some embodiments, the system processes information provided by a user to categorize them into a persona. Such user information may include, for example, demographics (e.g., age, gender, location, race, and so forth), medical conditions, and personality/psychometric survey answers.

In some embodiments, the system provides targeted educational content to the user according to the determined persona. In some embodiments, the targeted content is provided to increase the likelihood of the user engaging in advance care planning. In some embodiments, the targeted content is customized and provided, via a content recommendation engine, according to key motivators determined for each user. In some embodiments, the content recommendation engine employs a content model trained through machine learning to accurately categorize users into appropriate personas.

In some embodiments, the described targeted content system includes applications for mobile technology devices and desktop computing devices. In some embodiments, users interface with these applications in a healthcare setting, such as in a clinic or hospital, or through their personal computing device at home.

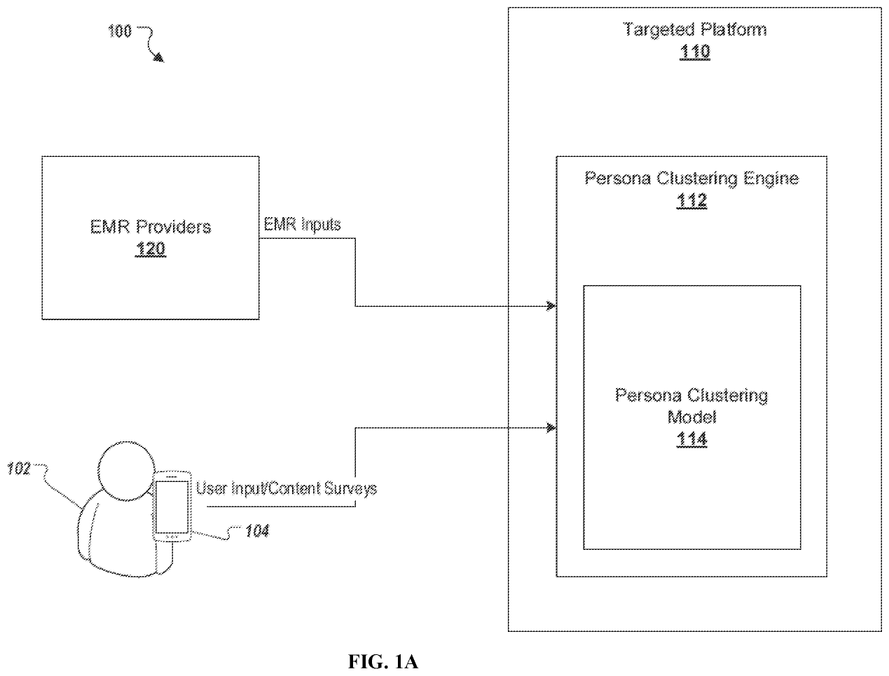

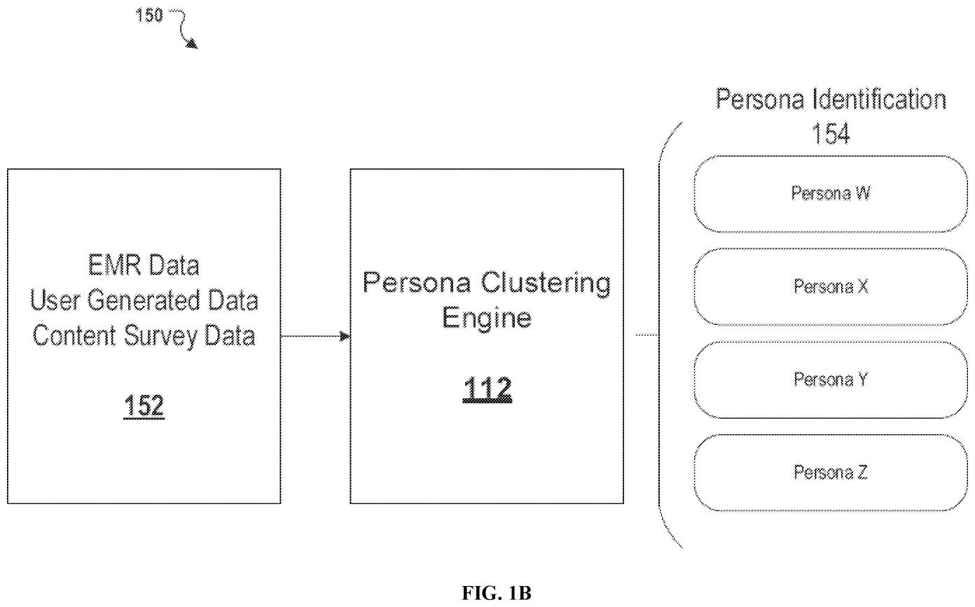

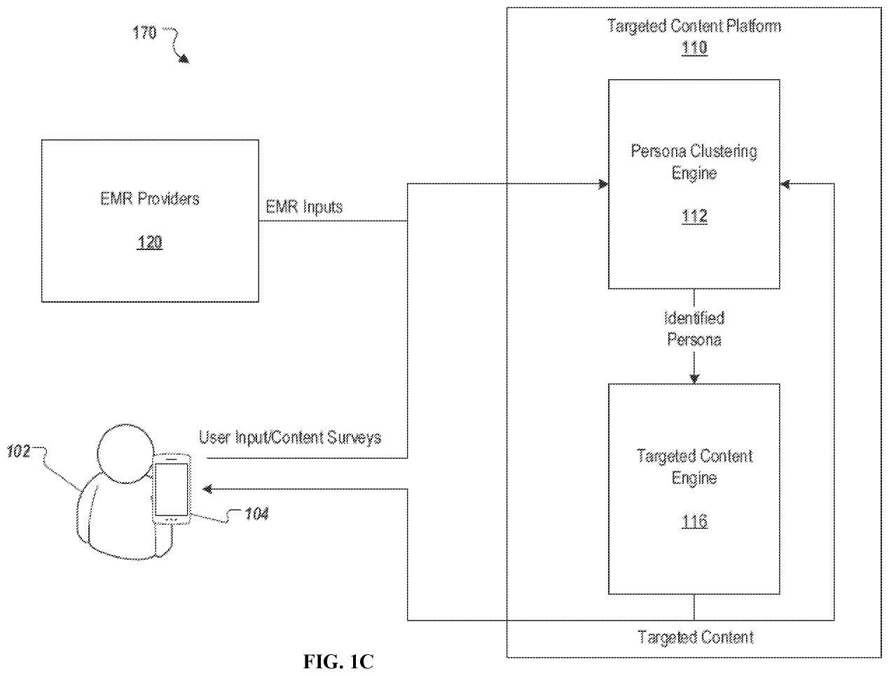

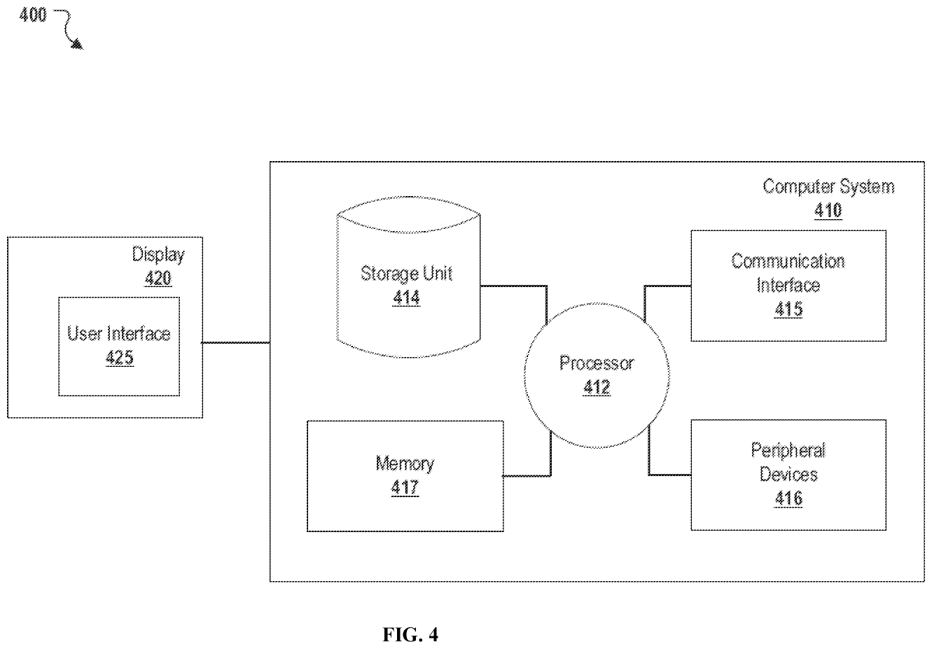

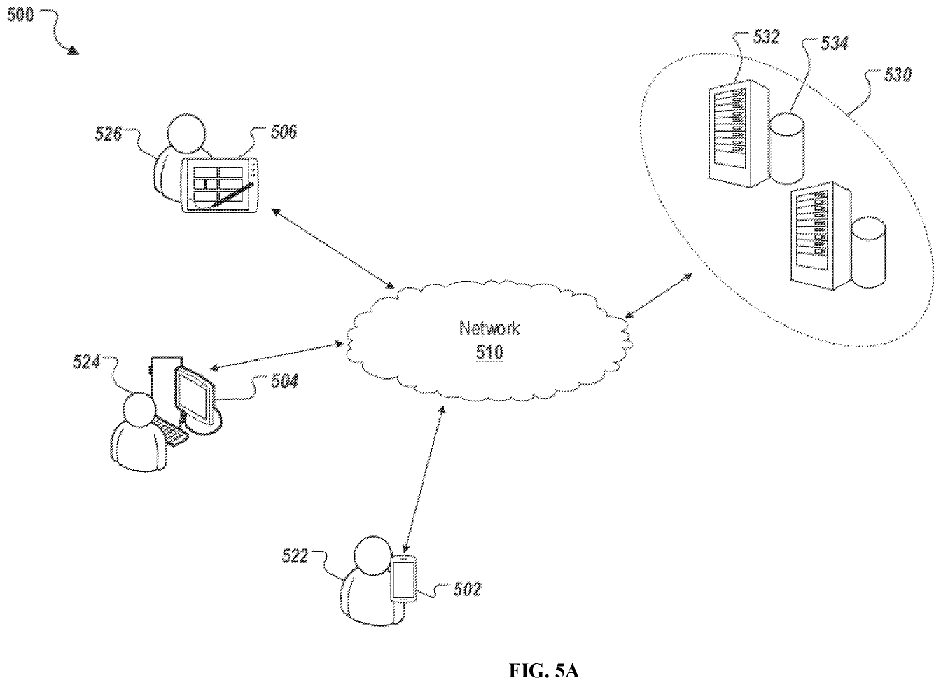

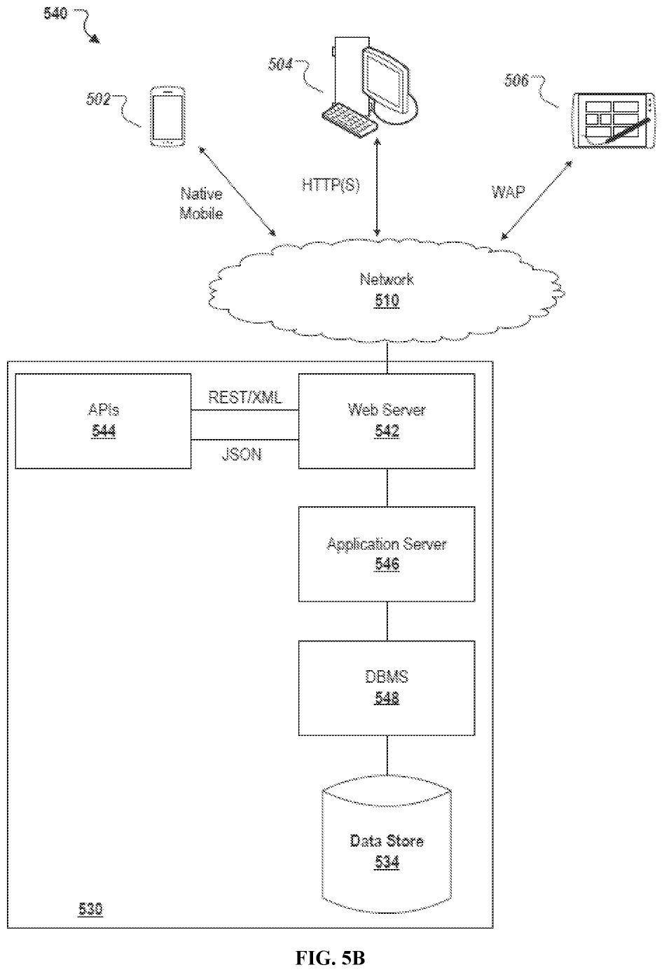

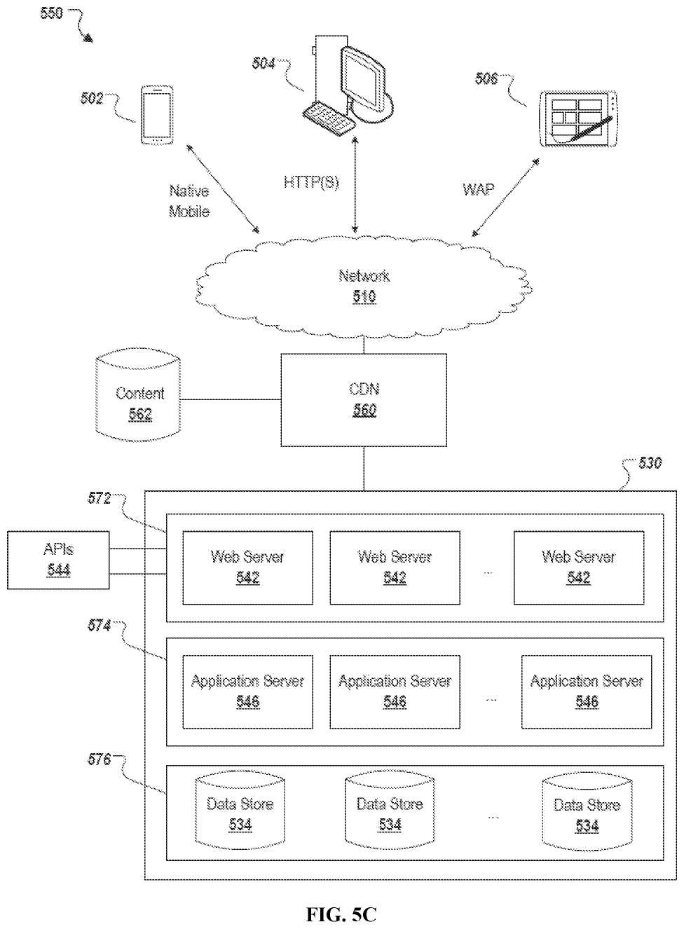

FIG. 1A depicts an example architecture 100 of the described targeted content system employed to train a persona clustering model 114. As depicted, the example architecture 100 includes users 102; user devices 104; a targeted content platform 110, which includes a persona clustering engine 112; and EMR providers 120. The user devices 104 can include any appropriate type of computing device. Such computing devices are described in greater detail below in the description of FIGS. 4 and 5A. The EMR providers 120 include one or more providers of EMR data.

In some embodiments, survey data as well as system inputs are collected from the users 102 (e.g., a via focus group) through the respective user devices 104 and provided to the persona clustering engine 112. Generally, the survey data includes information regarding a user's readiness to engage in advance care planning, or questions regarding user's values or personality. The users 102 may also provide information regarding ratings of various content with regards to how much the content would motivate them to engage in advance care planning. This content includes factors, such as, family, money, independence, and so forth. In some embodiments, EMR data is provided to the persona clustering engine 112 from the EMR providers 120. This EMR data includes, for example, demographic information, such as age, race, gender, and so forth as well as diagnosis information, and information about patients' visit and admission history and medical conditions. In some embodiments, the received data is recorded in a data store (not shown), which may be any suitable type of data store, such as a database. Data stores are described in greater detail below in the data store section. See FIGS. 5A-5C for various example architectures that may be employed to provide such services. The persona clustering engine 112 employs this received information to train persona clustering model 114 and to determine personas for the various clusters of users of the targeted content system.

As an example, an initial creation/training of a persona clustering model 114 by the persona clustering engine 112 may include receiving an initial set of user-generated data from a focus group of users 102 and receiving EMR data from the EMR providers 120 regarding the users 102 (or a set of similar identified similar users). The user-generated data may include, for example, an evaluation the readiness of a user to discuss advance care planning, answers to personality questions and a values assessment questionnaire, an evaluation of an engagement level of a user with various types of targeted content. The engagement level evaluation may include, for example, a rating of how much content related to family is a motivator to engage in advance care planning.

In some embodiments, the persona clustering engine 112 trains (e.g., via machine learning) a persona clustering model 114 by comparing EMR data and the user-generated data with responses to various targeted content provided by the users 102. The trained model 114 may be employed to determine a persona for later users of the targeted content system. In some embodiments, through the targeted content platform 110 targeted content (e.g., regarding advance care planning) is developed and assigned to each persona. Once a persona for a user has been determined based on the trained model 114, the targeted content can be customized for that user. For example, when users interact with the targeted content platform 110, the persona clustering engine 112 assess, via the trained model 114, which persona each user fit into, and a targeted content engine (see FIG. 1C) will then be able to deliver unique, targeted content to each user in real-time.

FIG. 1B depicts an example 150 various personas 154 determined by the persona clustering engine 112 according to the persona clustering model 114 (now shown) trained with received EMR data and user data 152. Four personas 154 (W, X, Y, and Z) are depicted in FIG. 1B for simplicity. It is contemplated, however, that embodiments of the present disclosure can be realized with any number of personas 154 according to the requirements for a set of targeted content provided by the described targeted content system.

FIG. 1C depicts an example architecture 170 of the described targeted content system employed to process received user data through a trained persona clustering model to determine at least one persona for a respective user. As depicted, the example architecture 200 includes the users 102; the user devices 104; the targeted content platform 110, which includes the persona clustering engine 112 and a targeted content engine 116; and the EMR providers 120. In some embodiments, the user 102 provides user-generated inputs regarding, for example, readiness to discuss advance care planning, personality questions, values (e.g., via an assessment questionnaire), personality, and so forth, to the persona clustering engine 112. In some embodiments, the targeted content platform 110 also queries the EMR providers 120 for EMR data such as diagnoses, demographics, visit history, medical conditions, and so forth for each of the users 102. This EMR data is also provided to the persona clustering engine 112.

In some embodiments, the persona clustering engine 112 processes the received data (the received user data and optionally, the received EMR data) to determine a persona for each user based on a set of personas (such as personas 154 depicted in FIG. 1B) associated with the specific targeted content provided by the targeted content platform 110.

In some embodiments, the determined persona is provided to the targeted content engine 116 where targeted content that is associated with the persona is customized for the user according to user provided data and optionally, the EMR data. For example, the targeted content engine 116 may generate specific educational content that aligns closely with the user's motivators and values based on the user's determined persona and provided content as some users are more motivated to engage in advance care planning by a desire to maintain independence, while other users are motivated by a desire to reduce family stress and anxiety. The educational content may include, for example, an interactive, video-based educational experience on advance care planning where, after each educational section, a user is asked a series of questions to understand what kind of care and values (care preferences) are important to that user if they are not able to make medical decisions for themselves. In some embodiments, the answers to these questions (e.g., the user's care preferences) be persisted in a data store as a user's profile and care plan. The answers can also be generated into a user-friendly advance directive.

In some embodiments, the determined targeted content is provided to the user 102 via the user device 104. In some embodiments, the targeted content may include specific content tailored for a user to, for example, increase engagement in advance care planning.

In some embodiments, the provided targeted content along with collected user-engagement and experience data is fed back to the persona clustering engine 112. In some embodiments, this content is employed by the persona clustering engine 112 to retrain the persona clustering model 114 (not shown). In some embodiments, the user-engagement and experience data includes web analytics, user survey results, and so forth.

### Example Processes

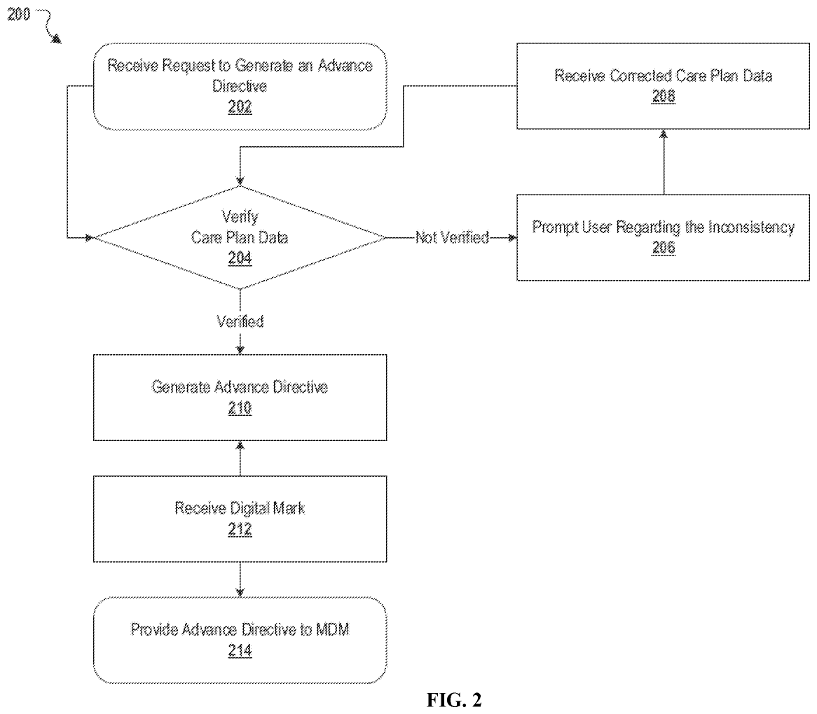

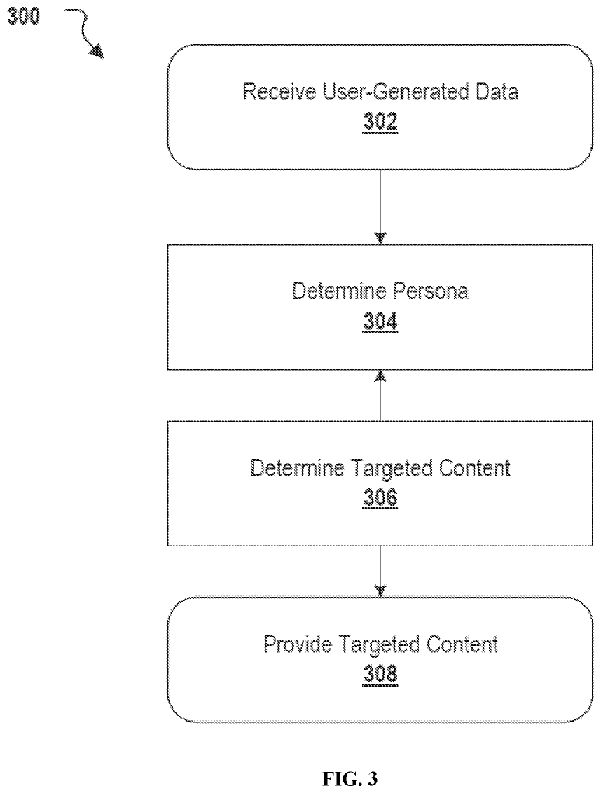

FIGS. 2 and 3 each depict a flowchart of example an example process 200 and 300 respectively, which can be implemented by embodiments of the present disclosure. The example processes 200 and 300 can be implemented by the components of the described targeted content system, such as described above in FIGS. 1A-1C. The example processes 200 and 300 generally shows in more detail how an advanced care directive may be generated via the described system.

For clarity of presentation, the description that follows generally describes the example processes 200 and 300 in the context of FIGS. 1A-1C, and 4-5C. However, it will be understood that the processes 200 and 300 may be performed, for example, by any other suitable system, environment, software, and hardware, or a combination of systems, environments, software, and hardware as appropriate. In some embodiments, various operations of the processes 200 and 300 can be run in parallel, in combination, in loops, or in any order.

For process 200 depicted in FIG. 2, at 202, a request to generate an advance directive is received from a user device, such as user device 104. At decision 204, care plan data associated with the user is verified by checking for completeness to ensure the necessary questions are answered and that the data follows appropriate logic. For example, if the user's care plan data includes information that they want to live as long as possible, no matter what, but also includes an indication that they do not want life-support, artificial nutrition, or dialysis, at 206, the inconsistency is provided to the user device to allow the user to provided clarification and consistent information. At 208, the corrected care plan data is received from the user device and the care plan data is verified at decision 204. When the care plan data is verified at decision 204, at 210, the advance directive is generated based on the verified care plan data according to any required legal parameters, such as the state where the users resides. At 212, the advance directive is provided to the user device. At 214, a digital mark (e.g., a signature) and, when necessary notarization, information is received from the user device. At 216, the signed advance directive is provided to the user's medical decision makers (MDMs) or physician(s).

For process 300 depicted in FIG. 3, at 302, user-generated data is received from a user-interface executing on a user device, such as user device 104 depicted in FIGS. 1A-1C. In some embodiments, the data comprised readiness, values, or personality of a user, From 302, the process 300 proceeds to 304.

At 304, the user-generated data is processed through a persona clustering model to determine a persona from a plurality of personas for the user. In some embodiments, the persona clustering model trained with previously received user-generated data for a plurality of other users. In some embodiments, the persona clustering model is retrained with the determined persona and the received user-generated data. In some embodiments, the persona is determined based on the readiness, the values, or the personality, provided for the user. In some embodiments, EMR data for the user is received via an API provided by an EMR provider. In some embodiments, the EMR data is processed through the persona clustering model to determine the persona. In some embodiments, the EMR data comprises diagnoses, demographics, or visit history for the user. In some embodiments, the user-generated data for the other users is received from the user-interface. In some embodiments, EMR data for the other users is received via the API provided the EMR provider. In some embodiments, the persona clustering model is trained with the received user-generated data for the other users and the EMR data for the other users. From 304, the process 300 proceeds to 306.

At 306, targeted content is determined for the user based on the persona. In some embodiments, the targeted content is determined based on key motivators to engage in advance care planning according to the determined persona. In some embodiments, the targeted content comprises education regarding advance care planning for the user, wherein the user can select care preferences. In some embodiments, after providing the targeted content, a selection of the care preferences is received from the user-interface. In some embodiments, the selected care preferences are persisted to a data store. In some embodiments, the selection of care preferences is verified. In some embodiments, an advanced directive is generated based on the selection of care preferences. In some embodiments, verifying the selection of care preferences comprises verifying the completeness of the selection of care preferences and verifying a logical consistency of the selection of care preferences. In some embodiments, the advanced directive determined based on the selection of care preferences is provided to the user-interface. In some embodiments, a digital mark of the user for the advanced directive is received from the user-interface. In some embodiments, the digital mark is verified. From 306, the process 300 proceeds to 308.

At 308, the targeted content customized for the user is provided to the user-interface. From 308, the process 300 ends.

### Processing Devices and Processors

In some embodiments, the platforms, systems, media, and methods described herein include a computer, or use of the same. In further embodiments, the computer includes one or more hardware central processing units (CPUs) or general purpose graphics processing units (GPGPUs) that carry out the device's functions. In still further embodiments, the computer comprises an operating system configured to perform executable instructions. In some embodiments, the computer is optionally connected a computer network. In further embodiments, the computer is optionally connected to the Internet such that it accesses the World Wide Web. In still further embodiments, the computer is optionally connected to a cloud computing infrastructure. In other embodiments, the computer is optionally connected to an intranet. In other embodiments, the computer is optionally connected to a data storage device.

In accordance with the description herein, suitable computers include, by way of non-limiting examples, server computers, desktop computers, laptop computers, notebook computers, sub-notebook computers, netbook computers, netpad computers, handheld computers, Internet appliances, mobile smartphones, tablet computers, and vehicles. Those of skill in the art will recognize that many smartphones are suitable for use in the system described herein. Those of skill in the art will also recognize that select televisions, video players, and digital music players with optional computer network connectivity are suitable for use in the system described herein. Suitable tablet computers include those with booklet, slate, and convertible configurations, known to those of skill in the art.

In some embodiments, the computer includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smart phone operating systems include, by way of non-limiting examples, Nokia® Symbian® OS, Apple® iOs®, Research In Motion® BlackBerry OS®, Google° Android®, Microsoft® Windows Phone® OS, Microsoft® Windows Mobile® OS, Linux®, and Palm® WebOS®.

In some embodiments, the device includes a storage or memory device. The storage or memory device is one or more physical apparatuses used to store data or programs on a temporary or permanent basis. In some embodiments, the device is volatile memory and requires power to maintain stored information. In some embodiments, the device is non-volatile memory and retains stored information when the computer is not powered. In further embodiments, the non-volatile memory comprises flash memory. In some embodiments, the volatile memory comprises dynamic random-access memory (DRAM). In some embodiments, the non-volatile memory comprises ferroelectric random access memory (FRAM). In some embodiments, the non-volatile memory comprises phase-change random access memory (PRAM). In other embodiments, the device is a storage device including, by way of non-limiting examples, compact disc read-only memory (CD-ROM), digital versatile disc (DVD), flash memory devices, magnetic disk drives, magnetic tapes drives, optical disk drives, and cloud computing-based storage. In further embodiments, the storage or memory device is a combination of devices such as those disclosed herein.

In some embodiments, the computer includes a display to send visual information to a user. In some embodiments, the display is a liquid crystal display (LCD). In further embodiments, the display is a thin film transistor liquid crystal display (TFT-LCD). In some embodiments, the display is an organic light emitting diode (OLED) display. In various further embodiments, on OLED display is a passive-matrix OLED (PMOLED) or active-matrix OLED (AMOLED) display. In some embodiments, the display is a plasma display. In other embodiments, the display is a video projector. In yet other embodiments, the display is a head-mounted display in communication with a computer, such as a virtual reality (VR) headset. In further embodiments, suitable VR headsets include, by way of non-limiting examples, HTC Vive, Oculus Rift, Samsung Gear VR, Microsoft HoloLens, Razer Open-Source Virtual Reality (OSVR), FOVE VR, Zeiss VR One, Avegant Glyph, Freefly VR headset, and the like. In still further embodiments, the display is a combination of devices such as those disclosed herein.

In some embodiments, the computer includes an input device to receive information from a user. In some embodiments, the input device is a keyboard. In some embodiments, the input device is a pointing device including, by way of non-limiting examples, a mouse, trackball, track pad, joystick, game controller, or stylus. In some embodiments, the input device is a touch screen or a multi-touch screen. In other embodiments, the input device is a microphone to capture voice or other sound input. In other embodiments, the input device is a video camera or other sensor to capture motion or visual input. In further embodiments, the input device is a Kinect, Leap Motion, or the like. In still further embodiments, the input device is a combination of devices such as those disclosed herein.

Computer control systems are provided herein that can be used to implement the platforms, systems, media, and methods of the disclosure. FIG. 4 depicts an example a computer system 410 that can be programmed or otherwise configured to implement platforms, systems, media, and methods of the present disclosure. For example, the computing device 410 can be programmed or otherwise configured to display or provided information to a user-interface or other applications provided by the described system.

In the depicted embodiment, the computing device 410 includes a CPU (also “processor” and “computer processor” herein) 412, which is optionally a single core, a multi core processor, or a plurality of processors for parallel processing. The computing device 410 also includes memory or memory location 417 (e.g., random-access memory, read-only memory, flash memory), electronic storage unit 414 (e.g., hard disk), communication interface 415 (e.g., a network adapter) for communicating with one or more other systems, and peripheral devices 416, such as cache, other memory, data storage or electronic display adapters. In some embodiments, the memory 417, storage unit 414, interface 415 and peripheral devices 416 are in communication with the CPU 412 through a communication bus (solid lines), such as a motherboard. The storage unit 414 comprises a data storage unit (or data repository) for storing data. The computing device 410 is optionally operatively coupled to a computer network, such as the network 510 depicted and described in FIG. 5A, with the aid of the communication interface 415. In some embodiments, the computing device 410 is configured as a back-end server deployed within the described system.

In some embodiments, the CPU 412 can execute a sequence of machine-readable instructions, which can be embodied in a program or software. The instructions may be stored in a memory location, such as the memory 417. The instructions can be directed to the CPU 412, which can subsequently program or otherwise configure the CPU 412 to implement methods of the present disclosure. Examples of operations performed by the CPU 412 can include fetch, decode, execute, and write back. In some embodiments, the CPU 412 is part of a circuit, such as an integrated circuit. One or more other components of the computing device 410 can be optionally included in the circuit. In some embodiments, the circuit is an application specific integrated circuit (ASIC) or a FPGA.

In some embodiments, the storage unit 414 stores files, such as drivers, libraries and saved programs. In some embodiments, the storage unit 414 stores user data, e.g., user preferences and user programs. In some embodiments, the computing device 410 includes one or more additional data storage units that are external, such as located on a remote server that is in communication through an intranet or the internet.

In some embodiments, the computing device 410 communicates with one or more remote computer systems through a network. For instance, the computing device 410 can communicate with a remote computer system. Examples of remote computer systems include personal computers (e.g., portable PC), slate or tablet PCs (e.g., Apple® iPad, Samsung® Galaxy Tab, etc.), smartphones (e.g., Apple® iPhone, Android-enabled device, Blackberry®, etc.), or personal digital assistants. In some embodiments, a user can access the computing device 410 via a network.

In some embodiments, the platforms, systems, media, and methods as described herein are implemented by way of machine (e.g., computer processor) executable code stored on an electronic storage location of the computing device 410, such as, for example, on the memory 417 or the electronic storage unit 414. In some embodiments, the CPU 412 is adapted to execute the code. In some embodiments, the machine executable or machine-readable code is provided in the form of software. In some embodiments, during use, the code is executed by the CPU 412. In some embodiments, the code is retrieved from the storage unit 414 and stored on the memory 417 for ready access by the CPU 412. In some situations, the electronic storage unit 414 is precluded, and machine-executable instructions are stored on the memory 417. In some embodiments, the code is pre-compiled. In some embodiments, the code is compiled during runtime. The code can be supplied in a programming language that can be selected to enable the code to execute in a pre-compiled or as-compiled fashion.

In some embodiments, the computing device 410 can include or be in communication with an electronic display 420. In some embodiments, the electronic display 420 provides a UI 425 that depicts various screen.

FIG. 5A depicts an example environment 500 that can be employed to execute embodiments of the present disclosure. The example system 500 includes computing devices 502, 504, 506, a back-end system 530, and a network 510. In some embodiments, the network 510 includes a local area network (LAN), wide area network (WAN), the Internet, or a combination thereof, and connects web sites, devices (e.g., the computing devices 502, 504, and 506) and back-end systems (e.g., the back-end system 530). In some embodiments, the network 510 includes the Internet, an intranet, an extranet, or an intranet or extranet that is in communication with the Internet. In some embodiments, the network 510 includes a telecommunication or a data network. In some embodiments, the network 510 can be accessed over a wired or a wireless communications link. For example, mobile computing devices (e.g., the smartphone device 502 and the tablet device 506), can use a cellular network to access the network 510.

In some examples, the users 522, 524, and 526 interact with the described system through a graphical user interface (GUI) or application that is installed and executing on their respective computing devices 502, 504, and 506. In some examples, the computing devices 502, 504, and 506 provide viewing data to screens with which the users 522, 524, and 526, can interact. In some embodiments, the computing devices 502, 504, 506 are sustainably similar to computing device 410 depicted in FIG. 4. The computing devices 502, 504, 506 may each include any appropriate type of computing device, such as a desktop computer, a laptop computer, a handheld computer, a tablet computer, a personal digital assistant (PDA), a cellular telephone, a network appliance, a camera, a smart phone, an enhanced general packet radio service (EGPRS) mobile phone, a media player, a navigation device, an email device, a game console, or an appropriate combination of any two or more of these devices or other data processing devices. Three user computing devices 502, 504, and 506 are depicted in FIG. 5A for simplicity. In the depicted example environment 500, the computing device 502 is depicted as a smartphone, the computing device 504 is depicted as a tablet-computing device, and the computing device 506 is depicted a desktop computing device. It is contemplated, however, that embodiments of the present disclosure can be realized with any of the appropriate computing devices, such as those mentioned previously. Moreover, embodiments of the present disclosure can employ any number of devices as required.

In the depicted example environment 500, the back-end system 530 includes at least one server device 532 and at least one data store 534. In some embodiments, the device 532 is sustainably similar to computing device 410 depicted in FIG. 4. In some embodiments, the back-end system 530 may include server-class hardware type devices. In some embodiments, the server device 532 is a server-class hardware type device. In some embodiments, the back-end system 530 includes computer systems using clustered computers and components to act as a single pool of seamless resources when accessed through the network 510. For example, such embodiments may be used in data center, cloud computing, storage area network (SAN), and network attached storage (NAS) applications. In some embodiments, the back-end system 530 is deployed using a virtual machine(s). In some embodiments, the data store 534 is a repository for persistently storing and managing collections of data. Example data stores that may be employed within the described system include data repositories, such as a database as well as simpler store types, such as files, emails, and so forth. In some embodiments, the data store 534 includes a database. In some embodiments, a database is a series of bytes or an organized collection of data that is managed by a database management system (DBMS).

In some embodiments, the at least one server system 532 hosts one or more computer-implemented services, such as described above, provided by the described system that users 522, 524, and 526 can interact with using the respective computing devices 502, 504, and 506.

FIG. 5B depicts an example application provision system 540 that can be provided through an environment, such as the example environment 500 and employed to execute embodiments of the present disclosure. As depicted, the example application provision system 540 includes the back-end system 530 configured to include one or more data stores 534 accessed by a DBMS 548. Suitable DBMSs include Firebird, MySQL, PostgreSQL, SQLite, Oracle Database, Microsoft SQL Server, IBM DB2, IBM Informix, SAP Sybase, SAP Sybase, Teradata, and the like. As depicted, the example application provision system 540 includes the back-end system 530 configured to include one or more application severs 546 (such as Java servers, .NET servers, PHP servers, and the like) and one or more web servers 542 (such as Apache, IIS, GWS and the like). The web server(s) 542 optionally expose one or more web services via an API 544 via the network 510. In some embodiments, the example application provision system 540 provides browser-based or mobile native UIs to the computing devices 502, 504, 506. Optionally, as described above, webservices may be deployed to a PaaS, such as Heroku.

FIG. 5C depicts an example cloud-based architecture of an application provision system 550 that can be provided through an environment, such as the example environment 500, and employed to execute embodiments of the present disclosure. The application provision system 550 includes the back-end system 530 configured to include elastically load balanced, auto-scaling web server resources 572, application server resources 574, as well as synchronously replicated stores 576. In some embodiment, of the example cloud-based architecture of an application provision system 550, content 562 of services are provided through a content delivery network (CDN) 560 coupled with the back-end system 530. In some embodiments, a CDN is a geographically distributed network of proxy servers and respective data centers that provides high availability and high performance through distributing the service spatially relative to the receiving devices, such as commuting devices 502, 504, and 506.

### Non-Transitory Computer Readable Storage Medium

In some embodiments, the platforms, systems, media, and methods disclosed herein include one or more non-transitory computer readable storage media encoded with a program including instructions executable by the operating system of an optionally networked computer. In further embodiments, a computer readable storage medium is a tangible component of a computer. In still further embodiments, a computer readable storage medium is optionally removable from a computer. In some embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In some cases, the program and instructions are permanently, substantially permanently, semi-permanently, or non-transitorily encoded on the media.

### Computer Program

In some embodiments, the platforms, systems, media, and methods disclosed herein include at least one computer program, or use of the same. A computer program includes a sequence of instructions, executable in the computer's CPU, written to perform a specified task. Computer readable instructions may be implemented as program modules, such as functions, objects, API, data structures, and the like, that perform particular tasks or implement particular abstract data types. Considering the disclosure provided herein, those of skill in the art will recognize that a computer program may be written in various versions of various languages.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Machine Learning

In some embodiments, machine learning algorithms are employed to build a model to determine quantifiable measures of dyadic ties between the individuals. In some embodiments, machine learning algorithms are employed to build a model to determine the filter relevant or chorological contact information for a user. Examples of machine learning algorithms may include a support vector machine (SVM), a naive Bayes classification, a random forest, a neural network, deep learning, or other supervised learning algorithm or unsupervised learning algorithm for classification and regression. The machine learning algorithms may be trained using one or more training datasets. For example, previously received contextual data may be employed to train various algorithms. Moreover, as described above, these algorithms can be continuously trained/retrained using real-time user data as it is received. In some embodiments, the machine learning algorithm employs regression modelling where relationships between variables are determined and weighted. In some embodiments, the machine learning algorithm employ regression modelling, wherein relationships between predictor variables and dependent variables are determined and weighted.

### Web Application

In some embodiments, a computer program includes a web application. Considering the disclosure provided herein, those of skill in the art will recognize that a web application, in various embodiments, utilizes one or more software frameworks and one or more database systems. In some embodiments, a web application is created upon a software framework such as Microsoft® .NET or Ruby on Rails (RoR). In some embodiments, a web application utilizes one or more database systems including, by way of non-limiting examples, relational, non-relational, object oriented, associative, and eXtensible Markup Language (XML) database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or XML. In some embodiments, a web application is written to some extent in a presentation definition language such as Cascading Style Sheets (CSS). In some embodiments, a web application is written to some extent in a client-side scripting language such as Asynchronous JavaScript and XML (AJAX), Flash® ActionScript, JavaScript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. In some embodiments, a web application includes a media player element. In various further embodiments, a media player element utilizes one or more of many suitable multimedia technologies including, by way of non-limiting examples, Adobe® Flash HTML 5, Apple® QuickTime®, Microsoft® Silverlight®, Java™, and Unity®.

### Mobile Application

In some embodiments, a computer program includes a mobile application provided to a mobile computer. In some embodiments, the mobile application is provided to a mobile computer at the time it is manufactured. In other embodiments, the mobile application is provided to a mobile computer via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C#, Objective-C, Java™, JavaScript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator , Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Google® Play, Chrome Web Store, BlackBerry® App World, App Store for Palm devices, App Catalog for webOS, Windows® Marketplace for Mobile, Ovi Store for Nokia® devices, Samsung® Apps, and Nintendo® DSi Shop.

### Standalone Application

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB .NET, or combinations thereof. Compilation is often performed, at least in part, to create an executable program. In some embodiments, a computer program includes one or more executable complied applications.

### Software Modules

In some embodiments, the platforms, systems, media, and methods disclosed herein include software, server, or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location.

### Data Stores

In some embodiments, the platforms, systems, media, and methods disclosed herein include one or more data stores. In view of the disclosure provided herein, those of skill in the art will recognize that data stores are repositories for persistently storing and managing collections of data. Types of data stores repositories include, for example, databases and simpler store types, or use of the same. Simpler store types include files, emails, and so forth. In some embodiments, a database is a series of bytes that is managed by a DBMS. Many databases are suitable for receiving various types of data, such as weather, maritime, environmental, civil, governmental, or military data. In various embodiments, suitable databases include, by way of non-limiting examples, relational databases, non-relational databases, object-oriented databases, object databases, entity-relationship model databases, associative databases, and XML databases. Further non-limiting examples include SQL, PostgreSQL, MySQL, Oracle, DB2, and Sybase. In some embodiments, a database is internet-based. In some embodiments, a database is web-based. In some embodiments, a database is cloud computing-based. In some embodiments, a database is based on one or more local computer storage devices.

While preferred embodiments of the present invention have been shown and described herein, it will be obvious to those skilled in the art that such embodiments are provided by way of example only. Numerous variations, changes, and substitutions will now occur to those skilled in the art without departing from the invention. Various alternatives to the embodiments of the invention described herein may be employed in practicing the invention.

What is claimed is:

1. A computer-implemented method for providing targeted content based on a determined persona, the method being executed by one or more processors and comprising:
receiving, from a user-interface, user-generated data comprising readiness, values, or personality, of a user;
processing the user-generated data through a persona clustering model to determine a persona from a plurality of personas for the user, the persona clustering model trained with previously received user-generated data for a plurality of other users;
determining targeted content for the user based on the persona; and
providing, to the user-interface, the targeted content customized for the user.

2. The method of claim 1, wherein the persona clustering model is retrained with the determined persona and the received user-generated data.

3. The method of claim 1, wherein the persona is determined based on the readiness, the values, or the personality, provided for the user.

4. The method of claim 1, wherein the targeted content is determined based on key motivators to engage in advance care planning according to the determined persona.

5. The method of claim 4, wherein the targeted content comprises education regarding advance care planning for the user, wherein the user can select care preferences.

6. The method of claim 5, comprising:
after providing the targeted content, receiving, from the user-interface, a selection of the care preferences; and
persisting the selected care preferences to a data store.

7. The method of claim 6, comprising:
verifying the selection of care preferences, and
generating an advanced directive based on the selection of care preferences.

8. The method of claim 7, wherein verifying the selection of care preferences comprises verifying the completeness of the selection of care preferences and verifying a logical consistency of the selection of care preferences.

9. The method of claim 7, comprising:
providing, to the user-interface, the advanced directive determined based on the selection of care preferences;
receiving, from the user-interface, a digital mark of the user for the advanced directive; and
verifying the digital mark.

10. The method of claim 1, comprising:
receiving, via an application program interface (API) provided by an electronic medical records (EMR) provider, EMR data for the user; and
processing the EMR data through the persona clustering model to determine the persona.

11. The method of claim 10, wherein the EMR data comprises diagnoses, demographics, or visit history for the user.

12. The method of claim 11, comprising:
receiving, from the user-interface, the user-generated data for the other users;
receiving, via the API provided the EMR provider, EMR data for the other users; and
training the persona clustering model with the received user-generated data for the other users and the EMR data for the other users.

13. One or more non-transitory computer-readable storage media coupled to one or more processors and having instructions stored thereon which, when executed by the one or more processors, cause the one or more processors to perform operations comprising:
receiving, from a user-interface, user-generated data comprising readiness, values, or personality, of a user;
processing the user-generated data through a persona clustering model to determine a persona from a plurality of personas for the user, the persona clustering model trained with previously received user-generated data for a plurality of other users;
determining targeted content for the user based on the persona; and
providing, to the user-interface, the targeted content customized for the user.

14. A targeted content system, comprising:
a user-interface;
one or more processors; and
a computer-readable storage device coupled to the one or more processors and having instructions stored thereon which, when executed by the one or more processors, cause the one or more processors to perform operations comprising:
receiving, from the user-interface, user-generated data comprising readiness, values, or personality, of a user;
processing the user-generated data through a persona clustering model to determine a persona from a plurality of personas for the user, the persona clustering model trained with previously received user-generated data for a plurality of other users;
determining targeted content for the user based on the persona; and
providing, to the user-interface, the targeted content customized for the user.

15. The system of claim 14, wherein the targeted content comprises education regarding advance care planning for the user, wherein the user can select care preferences, and wherein the operations comprise:
after providing the targeted content, receiving, from the user-interface, a selection of the care preferences;
persisting the selected care preferences to a data store;
verifying the selection of care preferences by verifying the completeness of the selection of care preferences and verifying a logical consistency of the selection of care preferences;
generating an advanced directive based on the selection of care preferences;
providing, to the user-interface, the advanced directive determined based on the selection of care preferences;
receiving, from the user-interface, a digital mark of the user for the advanced directive; and
verifying the digital mark.

16. The system of claim 14, wherein the persona clustering model is retrained with the determined persona and the received user-generated data.

17. The system of claim 14, wherein the persona is determined based the readiness, the values, or the personality, provided for the user.

18. The system of claim 14, wherein the targeted content is determined based on key motivators to engage in advance care planning according to the determined persona.

19. The system of claim 14, wherein the operations comprise:
receiving, via an application program interface (API) provided by an electronic medical records (EMR) provider, EMR data for the user; and
processing the EMR data through the persona clustering model to determine the persona.

20. The system of claim 19, wherein the EMR data comprises diagnoses, demographics, or visit history for the user, and wherein the operations comprise:
receiving, from the user-interface, the user-generated data for the other users;
receiving, via the API provided the EMR provider, EMR data for the other users; and
training the persona clustering model with the received user-generated data for the other users and the EMR data for the other users.
